(19) United States
(12) Patent Application Publication
(10) Pub. No.: US 2012/0288961 A1
(43) Pub. Date: Nov. 15, 2012

(54) CAPILLARITY-BASED DEVICES FOR PERFORMING CHEMICAL PROCESSES AND ASSOCIATED SYSTEMS AND METHODS

(75) Inventors: Paul Yager, Seattle, WA (US); Barry R. Lutz, Seattle, WA (US); Elain S. Fu, Seattle, WA (US); Peter C. Kauffman, Bainbridge Island, WA (US)

(73) Assignee: University of Washington, Seattle, WA (US)

(21) Appl. No.: 13/518,365

(22) PCT Filed: Dec. 21, 2010
(86) PCT No.: PCT/US2010/061675

§ 371 Date: Jun. 21, 2012

Related U.S. Application Data
(60) Provisional application No. 61/289,156, filed on Dec. 22, 2009.

(51) Int. Cl. B01L 3/00; G01N 33/558

(52) U.S. Cl. 436/501; 422/502

(57) ABSTRACT

The present technology is directed to capillarity-based devices for performing chemical processes and associated system and methods. In one embodiment, for example, a device can include a base configured to receive one or more fluids, a porous wick carried by the base portion, and a flow-metering element along the porous wick to modify a rate or volume of fluid flow along the porous wick. The porous wick can comprise a first pathway, a second pathway, and an intersection at which the first pathway and the second pathway converge. Input ends of the first and second pathways can be wettably distinct. Upon wetting of the input ends, fluid is configured to travel by capillary action along each pathway. The device may also include volume-metering features configured to automatically and independently control or modify a volume of fluid flow along one or more pathways of the porous wick.

### CROSS-REFERENCE TO RELATED APPLICATION

This application claims priority to U.S. Provisional Patent Application No. 61/289,156, filed Dec. 22, 2009, and incorporated herein by reference in its entirety.

### TECHNICAL FIELD

The present technology generally related to capillarity-based devices for performing chemical processes and associated systems and methods. In particular, several embodiments are directed toward a capillarity-based device that makes use of a flow-metering element and/or a volume-metering feature on a porous membrane to perform microfluidic analyses.

### BACKGROUND

Porous membranes are often used in conventional lateral flow and flow-through cartridges, in which flow of fluid occurs by wicking through the membrane (either laterally or transversely) onto an absorbent pad. Immunoassays take advantage of porous wick systems to measure and analyze analyte samples. The dependence on wicking to generate flow greatly limits control over assay conditions. Specifically, lateral flow assays are often limited to a single step in which sample (and buffer) is added to the sample pad, and the sample flows by capillary action (i.e., wicking) along the pad. Capillarity provides the force needed to provide a nearly continuous flow of fluid from one point to another, causing reagents stored in dry form to be transported along the device and to pass over regions that contain immobilized capture molecules. These devices are restricted to simple one-shot detection chemistries like colored nanoparticles that do not provide the sensitivity possible with multistep-detection chemistries, such as enzymatic amplification. They are also rarely quantitative.

Microfluidic systems that include open fluid channels for the flow of buffers, samples, and reagents can inherently be made much more sophisticated, and it is possible to use them to carry out a very large number of fluid-processing steps. Such microfluidic systems usually incorporate a complex disposable, which leads to unavoidably high per-test manufacturing costs and the need for expensive external pumps and valves to move fluids. While microfluidic devices can inherently be very flexible in the functions that they perform, they are also inherently complicated and expensive. Additionally, the devices that have been made that support complex function are usually quite complex themselves. For example, some polymeric laminate cartridges currently developed contain as many as 23 different layers, each of which must be separately manufactured and bonded to the others.

### BRIEF DESCRIPTION OF THE DRAWINGS

FIG. 1A is an isometric view of a capillarity-based device configured in accordance with an embodiment of the technology.

FIG. 1B is an exploded isometric view of the device of FIG. 1A.

FIG. 2A is a front view of a flow-metering element configured in accordance with an embodiment of the technology.

FIG. 2B is a front view of a flow-metering clement configured in accordance with an embodiment of the technology.

FIG. 2C is a series of time-lapsed front views of a pathway having a flow-metering element configured in accordance with an embodiment of the technology.

FIG. 2D is a series of time-lapsed front views of a pathway having a flow-metering element configured in accordance with an embodiment of the technology.

FIG. 3A is a series of time-lapsed front views of a pathway having a soluble barrier configured in accordance with an embodiment of the technology.

FIG. 3B is a series of time-lapsed front views of a capillarity-based device having a plurality of soluble barriers configured in accordance with an embodiment of the technology.

FIG. 3C is a series of time-lapsed front views of a pathway having a soluble restrictor configured in accordance with an embodiment of the technology.

FIG. 3D is a series of front views of pathways having soluble barriers or soluble restrictors configured in accordance with embodiments of the technology.

FIG. 4 is a series of time-lapsed front views of a capillarity-based device having a plurality of switchable barriers configured in accordance with an embodiment of the technology.

FIG. 5 is a series of time-lapsed front views of a volume-metering element configured in accordance with an embodiment of the technology.

FIG. 6 is a series of time-lapsed front views of a capillarity-based device having a volume-metering element configured in accordance with an embodiment of the technology.

FIG. 7A is a top view of a substrate having volume-metering absorbent pads configured in accordance with an embodiment of the technology.

FIG. 7B is a top view of the substrate of FIG. 7A placed on a capillarity-based device configured in accordance with an embodiment of the technology.

FIG. 8A is a series of time-lapsed front views of a capillarity-based device configured in accordance with an embodiment of the technology.

FIG. 8B is a front view of pre-wetted source pads for use with the device of FIG. 8A.

FIG. 8C is a timeline of reagent delivery from the pre-wetted source pads to a wicking pad of the device of FIG. 8A.

FIG. 9 is a front view of a capillarity-based device configured in accordance with an embodiment of the technology.

FIG. 10 is an isometric view of a capillarity-based device configured in accordance with an embodiment of the technology.

### DETAILED DESCRIPTION

The present technology describes various embodiments of devices for processing, analyzing, detecting, measuring and separating fluids. The devices can be used to perform these processes on a microfluidic scale, and with control over fluid and reagent transport. In one embodiment, for example, a device for performing chemical processes can include a base portion configured to receive one or more fluids, a porous wick carried by the base portion, and a flow-metering element along the porous wick to modify a rate or volume of fluid flow along the porous wick. The porous wick can comprise a first pathway, a second pathway, and an intersection at which the first pathway and the second pathway converge. Each pathway can comprise a length defined by an input end and an output end and a width defined by two sides. The input ends of the first pathway and the second pathway can be wettably distinct. Upon wetting of the input ends, fluid is configured to travel by capillary action along each pathway. The device can also include volume-metering features configured to automatically and independently control or modify a volume of fluid flow along one or more pathways of the porous wick.

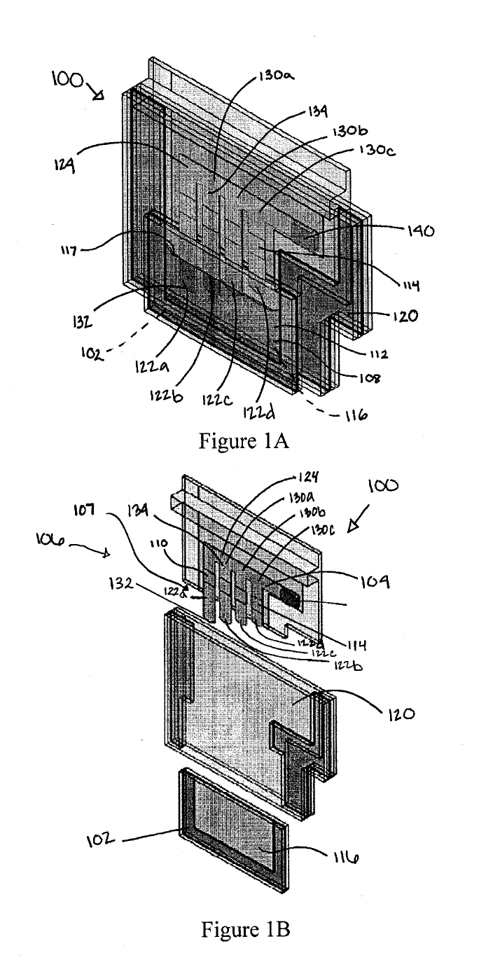

Specific details of several embodiments of the technology are described below with reference to FIGS. 1A-10. Other details describing well-known structures and systems often associated with capillarity-based devices, biomedical diagnostics, immunoassays, etc. have not been set forth in the following disclosure to avoid unnecessarily obscuring the description of the various embodiments of the technology. Many of the details, dimensions, angles, and other features shown in the Figures are merely illustrative of particular embodiments of the technology. Accordingly, other embodiments can have other details, dimensions, angles, and features without departing from the spirit or scope of the present technology. A person of ordinary skill in the art, therefore, will accordingly understand that the technology may have other embodiments with additional elements, or the technology may have other embodiments without several of the features shown and described below with reference to FIGS. 1A-10.

As used herein, the term “wick” refers to a material over which fluid can travel by capillary action. Typically, the wick is a porous membrane. Representative examples of such porous membranes include paper, nitrocellulose, nylon, and many other materials recognized by those skilled in the art as capable of serving as a wick in the context of the present technology. The wick can be two-dimensional or three-dimensional (when considering its height in addition to its length and width). In some embodiments, the wick is a single layer, while in other embodiments, the wick comprises two or more layers of membrane.

As used herein, the term “pathway” or “leg” refers to an elongated wick having a length greater than its width. Because the pathway is membranous, fluid traverses the pathway via capillary action or wicking The width of the pathway is defined by sides or edges that limit the area of the pathway that can be traversed by fluid. Pathways can be patterned on a wick either by cutting the wick or by deposition of an insoluble barrier to create the desired configuration of pathways and pathway intersection(s).

As used herein, the term “wettably distinct” means being capable of being wetted by contact with separate fluids without mixing of the fluids at the point of initial wetting. For example, two input legs are wettably distinct if they are physically separated so that each leg could be brought into contact with a separate fluid reservoir. Pathways can be made wettably distinct by a variety of means including, but not limited to, separation via distinct edges (e.g., cut as separate pathways) and separation via an impermeable barrier.

### A. Capillarity-Based Devices and Associated Systems and Methods

FIG. 1A is an isometric view of a capillarity-based device or analyzer 100 configured in accordance with an embodiment of the technology, and FIG. 1B is an exploded isometric view of the device 100. Referring to FIGS. 1A and 1B together, the device 100 includes a base or housing 102, a porous wick 104, one or more flow-metering elements 106, and volume-metering features 107. The flow-metering element 106 and volume-metering features 107 are configured to automatically and independently control or modify a rate or volume of fluid flow along the porous wick 104. Further details regarding the device 100, the flow-metering element 106, and the volume-metering features 107 are described below.

The base or housing 102 can be configured to receive one or more fluids 108 (FIG. 1A), such as a sample, inert fluid, or reagent. In some embodiments, the base 102 can include one or more fluid wells or portals 116 configured to receive the fluids 108. Individual wells 116 can contain the same or different fluids. In other embodiments, these wells 116 can be absent and fluid can be supplied to the device 100 by other methods described in further detail below. The base 102 can be configured to support or carry the porous wick 104 in a vertical or horizontal orientation, or at an angle between the vertical or horizontal planes. In one embodiment, for example, the base 102 carries the wick 104 at an angle from about 45 degrees to about 90 degrees relative to the horizontal plane. The base 102 may further include an enclosure 120 that at least partially covers or surrounds the wick 104. In addition to providing structural support to the wick 104, the base 102 can further serve to protect the wick 104 from contamination, prevent evaporation of fluids 108 from the wick 104, and control humidity or other environmental conditions. The base 102 can be made of plastic, metal, glass, other materials, or a combination of materials.

In the illustrated embodiment, the wick 104 includes a plurality of pathways or legs 122a-122d (collectively 122). Each pathway 122a-d has an input end 132, an output end 134, and a length between the input end 132 and the output end 134. Each pathway 122a-d can further include a width defined by two sides. The input ends 132 of the individual pathways 122a-d can be wettably distinct from one another. Pathways 122 or portions thereof can be generally straight or curved. In some embodiments of the technology, for example, at least one pathway 122 is nonlinear. A serpentine pathway 122, for example, can zigzag via a series of curves, hairpin turns, sharp angles, or combinations thereof.

The pathways 122a-d intersect and converge into a common pathway 124. Two or more of the pathways can converge at the same or different locations or intersections 130a-130c (collectively 130) along the wick 104. Intersections 130 between pathways 122 can be at right angles or at larger or smaller angles. In some embodiments, for example, there may be a primary or first pathway 122a and a primary or first intersection 130a at which the primary or first pathway 122a converges with a secondary or second pathway 122b. In other embodiments, not all the pathways 122 need necessarily intersect. In still other embodiments, the merged pathways 122 can diverge into at least two pathways having wettably distinct output ends 134. In this latter embodiment, larger particles can be separated from a sample fluid in order to facilitate analysis of smaller analyte particles. In various embodiments that will be discussed in more detail below, fluid(s) 108 can travel and/or admix along pathways 122 and through intersections 130 simultaneously or sequentially.

The wick 104 can be composed of various materials including, for example, paper. In some embodiments, the wick 104 can be composed of backed nitrocellulose cut by a CO₂ laser. In some embodiments, the wick 104 have a thickness of about 0.120 mm or greater. The wick 104 can be given a desired pathway configuration by printing onto the wick 104 or by cutting the wick 104. Cutting the wick 104 can be performed by any of several low- or high-throughput methods, including computer-controlled knife cutters. Patterning of the pathways 122 on the wick 104 can be achieved, for example, by cutting the wick 104 and/or by treating the wick 104 to create pathways 122 that can be traversed by fluid 108. In one embodiment, for example, sides of the pathways 122 may be defined by the edge of the porous wick 104. In another embodiment, the sides of the pathways 122 may be defined by an insoluble (e.g., impermeable, hydrophobic) barrier.

In several embodiments, the device 100 is devoid of a pump. The need for a pump may be obviated by a design that enables all fluid movement to be effected via capillary action. In operation, capillary force can be generated by the wick 104 itself (i.e., as the fluid initially wets the wick 104), or the capillary force can be generated by an absorbent pad (not shown) at the output end 134 of an individual pathway 122 or the common pathway 124. In one embodiment, the porous wick 104 can have a pore size of from about 200 nm to about 30 μm. In a particular embodiment, the pore size of the wick is from about 5 μm to about 20 μm. In some embodiments, the wick 104 can have an effective surface area about 300 times larger than a flat surface, allowing for increased measurement sensitivity and rapid diffusion. In other embodiments, however, the wick 104 can have different dimensions and/or arrangements.

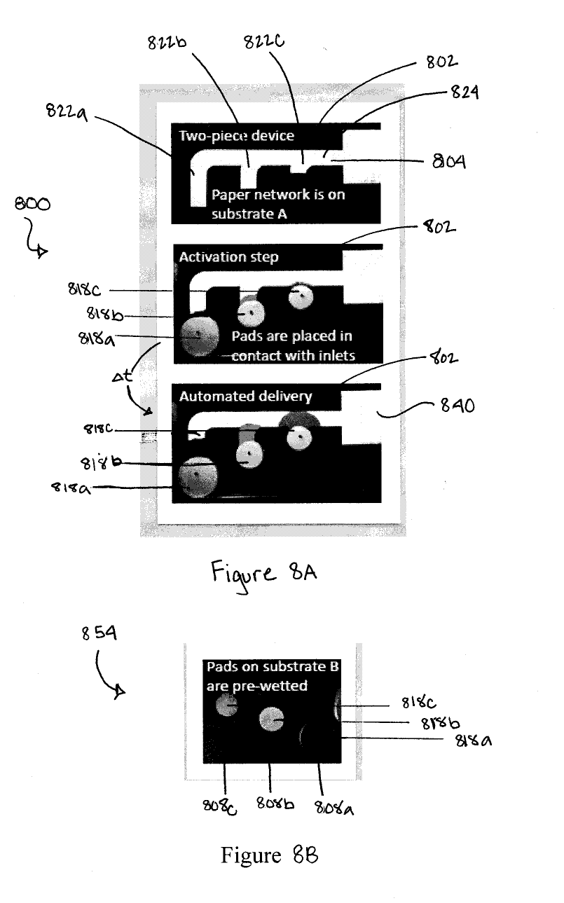

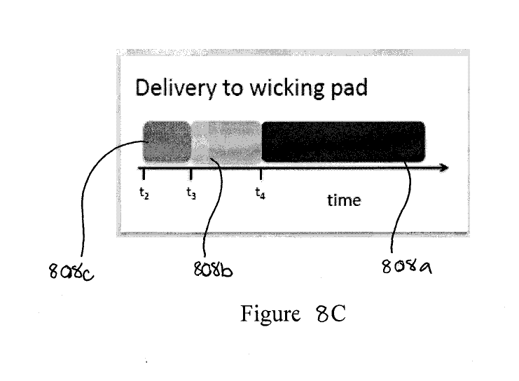

As noted above, the porous wick 104 is configured to wick one or more fluids (e.g., fluid(s) 108) from the input ends 132 toward the output ends 134 of the respective pathways 122 upon wetting of the pathways 122. In one embodiment, for example, the input ends 132 of the pathways 122 can contact the fluid 108 within the base 102, for instance, by submerging the wick 104 in the well 116 of the base 102. In another embodiment, a sample fluid can be applied to a pathway 122 before the wick 104 contacts the fluid reservoir 116. In this embodiment, the sample can flow solely by capillary action along the wick 104 or can be additionally pushed along the pathway 122 by upstream fluid 108 upon wetting the input end 134 of the pathway. In yet another embodiment, as discussed in more detail below with reference to FIGS. 7A-8C, the fluid(s) 108 can be placed directly on the wick 104 in the form of pre-wetted pads (not shown) or other means. In still further embodiments, the wick 104 can be wetted by a combination of these mechanisms. After wetting, the fluid(s) 108 travel along the wick 104 toward a detection region 140 where a chemical analysis is done or where results of the chemical analysis can be read by a user (not shown).

As mentioned previously, the device 100 can include one or more flow-metering elements 106. The individual flow-metering elements 106 are configured to control, regulate, or modify the fluid flow rate by altering the geometric characteristics (e.g., length, depth and width) or chemical characteristics (e.g., using materials of different composition) of the pathways 122 and/or by using chemical barriers and switches (not shown). The choice of wick 104 and/or pathway material, pore size, or surface treatment can affect the rate of fluid flow in the device 100. In some embodiments, for example, the wick 104 can be spotted with various chemistries to change the local surface chemistry. For example, the wick 104 can be spotted for permanent immobilization of capture molecules or for temporary storage of reagents that can be mobilized by flowing fluid.

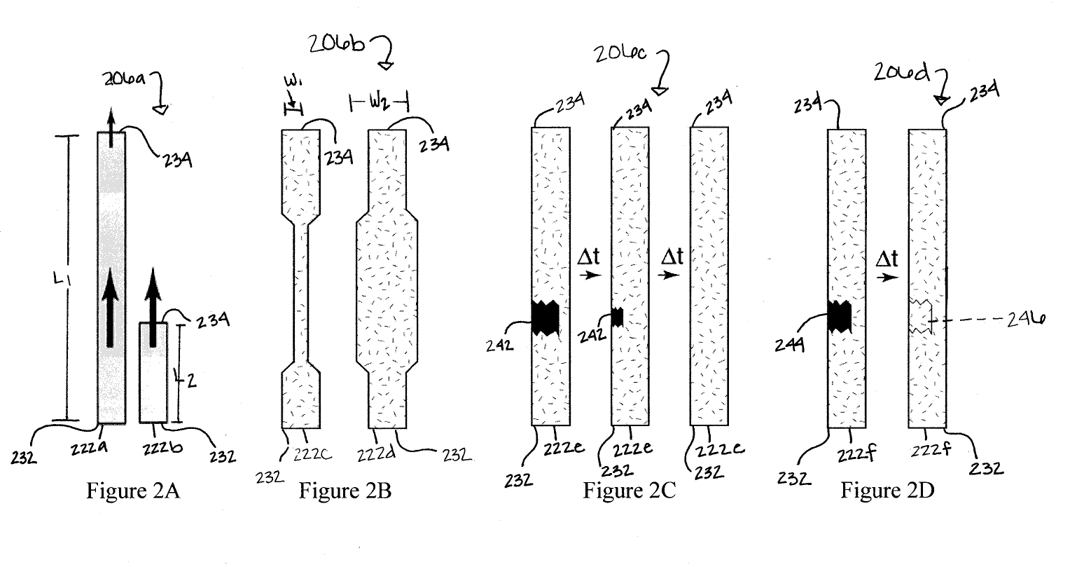

In the illustrated embodiment, the flow-metering element 106 includes pathways 122 having differing lengths with corresponding differing flow rates. In some embodiments, the flow-metering elements 106 can regulate timing of fluid arrival at one or more intersection points 130 or detection regions 140 in the device 100. Flow metering mechanisms will be described in further detail below with reference to FIGS. 2A-4.

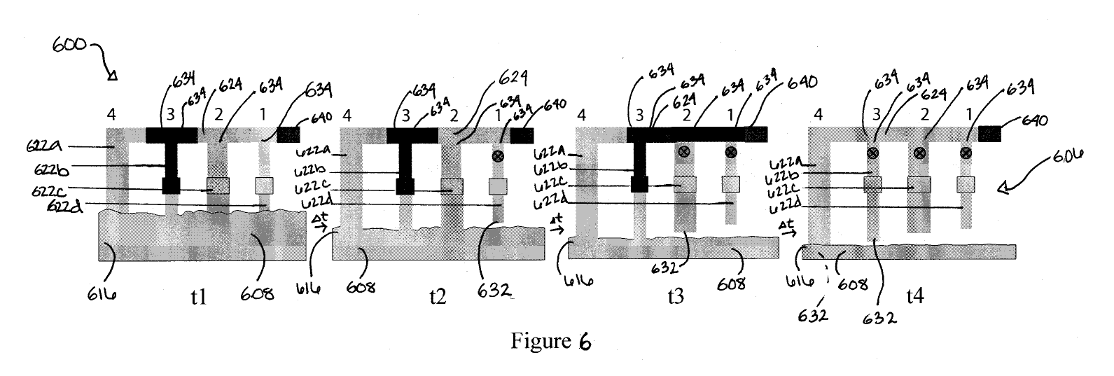

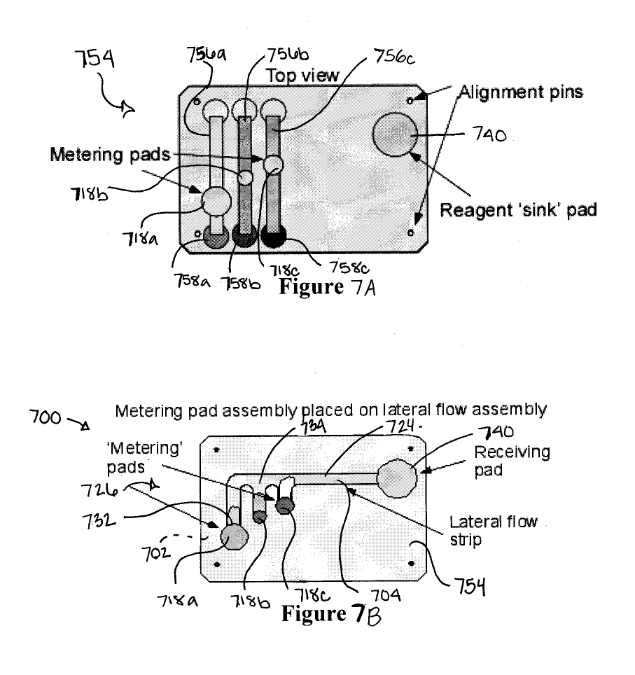

The device 100 can further include volume-metering features 107 that can control the volume and timing of fluid delivery to the input ends 132 of each pathway 122. In the illustrated embodiment, for example, the volume of fluid supplied and shut-off time for each pathway 122 is controlled by the relationship between the position of the input end 132 of each pathway 122 and a level of fluid 108 brought into contact with the input end 132, e.g., via submersion in the fluid-filled well 116. Specifically, the well 116 stops contacting, and therefore stops supplying fluid to, pathways or legs 122 that extend a shorter distance below a surface 117 of fluid 108 in the well 116 than legs 122 that extend a longer distance below the surface of fluid 117 in the well 116. Leg-limiting volume-metering features 107 are discussed in more detail below with reference to FIGS. 5 and 6. In other embodiments, other mechanisms can be used to control volume and timing. In one embodiment, for example, volume-limited fluid-delivery pads (discussed in more detail below with reference to FIGS. 7 and 8) can be used. In another embodiment, wells 116 can contain different volumes of source fluid 108 and can supply these different volumes to individual legs 122. In some embodiments, one pathway 122a can be wetted simultaneously with another pathway 122b. In other embodiments, however, the pathway 122a can begin or end wicking before or after another pathway 122b. In still further embodiments, the wick 104 (or portions of the wick) can have a limited fluid capacity, thereby serving as another means to regulate volume.

It will be appreciated that in the embodiment shown in FIGS. 1A and 1B, the flow-metering element 106 and volume-metering features 107 comprise similar features that perform similar functions in controlling/modifying the fluid flow along each pathway 122 of the wick 104. In other embodiments, however, the flow-metering element(s) and volume-metering feature(s) can comprise different components or features that function independently of each other to control/modify/regulate fluid flow.

The fluid traveling along each pathway 122 can include one or more samples (e.g., analytes) 110, reagents 114, indicators, binding/capture agents, and/or wash solutions 112. The sample 110 can include blood, urine, saliva or other bodily fluid, or other non-bodily fluids. In some embodiments, one or more reagents 114 can be placed on or embedded along the wick 104, either directly or on a substrate or pad (not shown). Reagents 114 can be spotted on the paper manually or using inkjet printing. In one embodiment approximately from about 3 μl to about 80 μl reagent 114 can be applied to the wick 104 or pad using a syringe or pipette. The reagents 114 can be immobilized on the wick 104 or can dissolve or become mobile upon contacting fluid 108 traveling along the wick. Depending upon reagent 114 placement on the wick 104 and upon the use of flow-metering elements 106 and/or volume-metering features 107, the fluid 108 entering the input ends 132 of the pathways 122 can make fluidic contact with different reagents 114 at differing times.

In some embodiments, the device 100 further includes a capture agent (not shown) that binds the analyte 110 disposed on the wick 104 downstream of the primary intersection 130a. Capture agents can be used for either direct or competitive assays to determine the presence and/or quantity of analyte 110 present in a sample. Typically, the device 100 further comprises the reagent 114 disposed on one of the secondary pathways (e.g., pathway 122b). The reagent 114 can be located downstream of the primary intersection 130a. The reagent 114 can interact with the analyte 110 and/or the capture agent, and can be mobilized upon contact with the fluid 108. The positioning of reagents 114 as well as pathways 122 that will be traversed by inert fluid (e.g., water, buffer) can be designed to create an appropriate series (sequential or simultaneous) of chemical interactions and washes that allow for all steps of a conventional assay, such as an immunoassay or a nucleic acid amplification and detection, to be performed on the wick 104. For example, the configuration of the pathways 122 and intersections 130 and the use of flow-metering elements 106 and/or volume-metering features 107 can be used to control the sequence of assay steps to be performed. In one example, a series of secondary pathways 122b/122c/122d merges via a series of intersections 130a/130b/130c into a single secondary pathway 124 that, in turn, intersects with the primary pathway 122a. Because the assay steps are all initiated by the fluid traversing the wick 104 via capillary action, the only necessary step to activate the entire series of assay steps is the initial contact between the input ends 132 of the pathways 122 and the fluid 108.

The device 100 can be used for analyzing, diffusing, detecting, filtering, processing, measuring and/or separating fluid samples 110. The device 100 may also be used for solid-phase assay and selective capture. The device 100 can be used to perform these processes on a microfluidic scale, and with control over fluid and reagent transport within the device 100.

### 1. Select Embodiments of Flow-Metering Elements

FIGS. 2A-4 illustrate various embodiments of flow-metering elements configured in accordance with embodiments of the technology. The flow-metering elements described below, for example, can be used with the device 100 (FIGS. 1A and 1B) or other suitable capillarity-based devices. The flow-metering elements of FIGS. 2A-2D are configured to modify a rate or volume of fluid flow along a porous wick (e.g., wick 104 of FIGS. 1A and 1B). The flow-metering elements may be located on one or more fluid pathways (e.g., pathways 222) and before or after an intersection point (e.g., intersection(s) 130) where the pathways converge. In some embodiments, the flow-metering elements can be an integral component of a porous wick. In other embodiments, however, the flow-metering elements may be removable from the wick. Further, in some instances, two or more of the following example flow-metering elements can be used in conjunction or separately to obtain the desired rate and volume of fluid flow.

FIGS. 2A and 2B, for example, are front views of flow-metering elements 206a and 206b, respectively, configured in accordance with embodiments of the technology. In particular, the flow-metering elements 206a and 206b illustrated in FIGS. 2A and 2B statically modify the rate or volume of fluid flow in a device by modifying a geometric characteristic of one or more pathways 222. For example, the flow-metering element 206a of FIG. 2A modifies the rate or volume of fluid flow by adjusting the length of one or more pathways 222, while the flow-metering element 206b of FIG. 2B modifies the rate or volume of fluid flow by adjusting the width of one or more pathways 222. The flow velocity along a pathway 222 is proportional to the capillary force generated at the fluid front (C×Wf) and inversely proportional to the sum of resistances for the flow path (Sum(Wi/Li)). Accordingly, the overall flow velocity for a pathway 222 can be adjusted by adjusting the pathway length and/or the pathway width.

Referring first to FIG. 2A, varied leg length is used to control the rate of fluid flow along pathways 222a and 222b. In a segment of constant width, movement of the fluid front advancing into a dry membrane depends on the resistance that the wet paper behind it presents to flow of the lengthening column of fluid. This resistance increases with length, so the further the front moves, the slower it moves, according to the Washburn equation:

L²=γDt/4μ′,

where L is distance moved by the fluid front, t is time, D is the average pore diameter, γ is surface tension, and μ is viscosity. In the illustrated embodiment, a first pathway 222a has an extended length L₁ that extends the time required for fluid to travel the pathway 222a relative to a second, shorter pathway 222b having length L₂.

Referring next the FIG. 2B, the flow-metering element 206b comprises first and second pathways 222c and 222d having differing widths. Resistance decreases as the width of the segment increases, so the speed of the front depends on the width of the segment behind it. Accordingly, fluid rate and output volume can be controlled by manipulating individual pathway widths. For example, in the illustrated embodiment, a portion of the first pathway 222c has a narrowed width W₁ that shortens the time required for fluid to travel the length of the narrowed portion of the pathway 222c relative to a second, wider pathway 222d having a width W₂.

FIGS. 2C and 2D include a series of time-lapsed front views of pathways having flow-metering elements configured thereon in accordance with additional embodiments of the technology. More specifically, FIGS. 2C and 2D illustrate pathways 222e and 222f having flow-metering elements 206c and 206d, respectively, that include a barrier placed along the fluid pathway. The barriers can be dissolvable or soluble (as illustrated in FIG. 2C) or switchable (as illustrated in FIG. 2D). Dissolvable and switchable barriers can be used to dynamically modify the rate at which fluid moves through the wick.

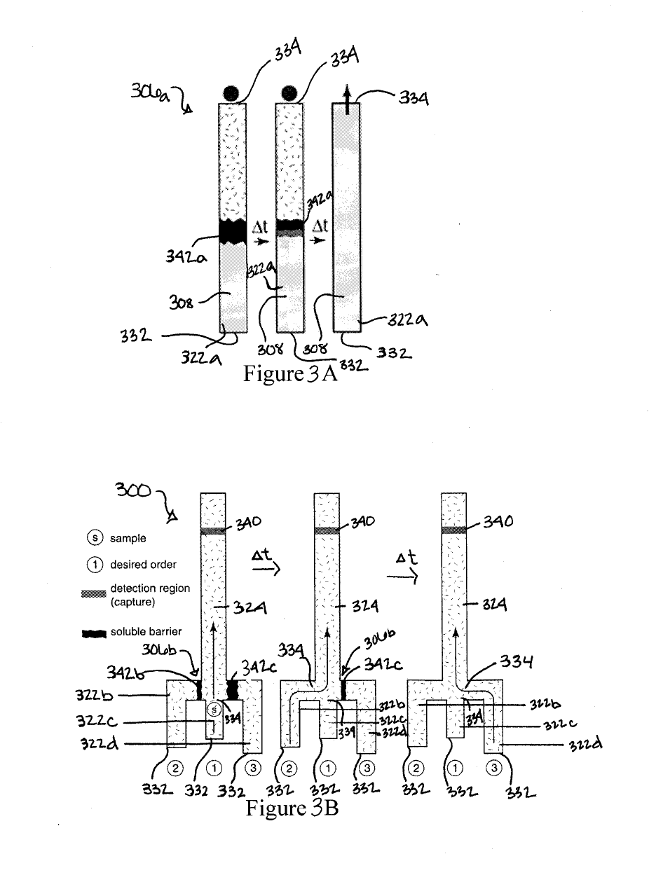

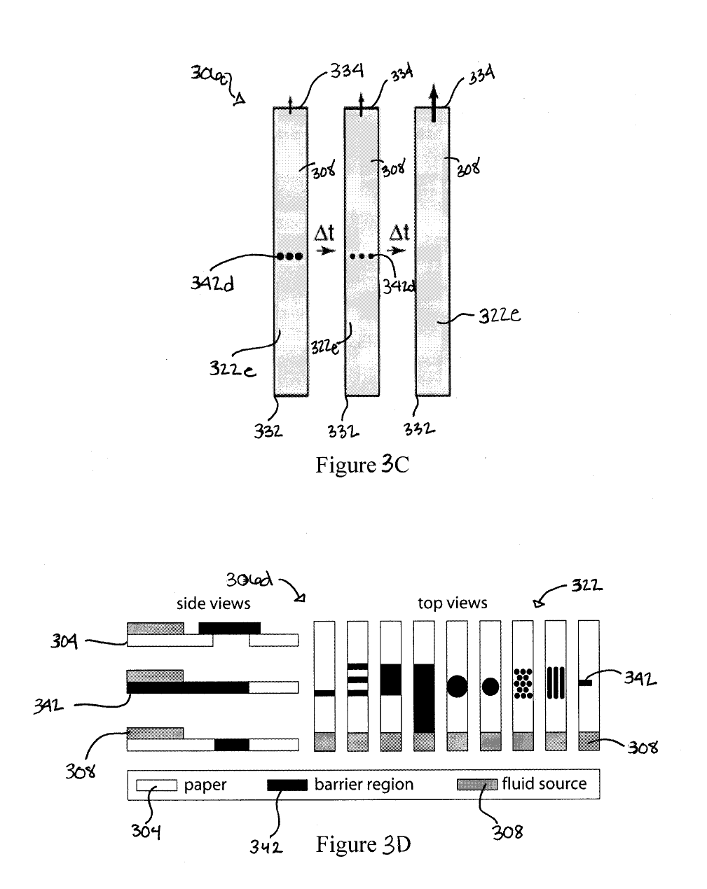

Referring first to FIG. 2C, the pathway 222e has a soluble barrier 242 disposed thereon and positioned to block or hinder fluid flow along the pathway 222e. In one embodiment, the barrier 242 can dissolve over time after contacting fluid in the wick. By controlling the length, width, and material of the barrier 242, the rate and volume of fluid flow along the pathway 222e can be regulated. Longer and/or wider barriers 242 can hinder fluid flow for a longer amount of time than shorter/narrower barriers 242. Furthermore, dissolvable barriers 242 can differ in their solubilities or dissolution rates, such as by use of differing materials, e.g., salt, sugar, etc., or in differing mixtures or concentrations of materials. Soluble barriers 242 will be discussed further below with reference to FIGS. 3A-3D.

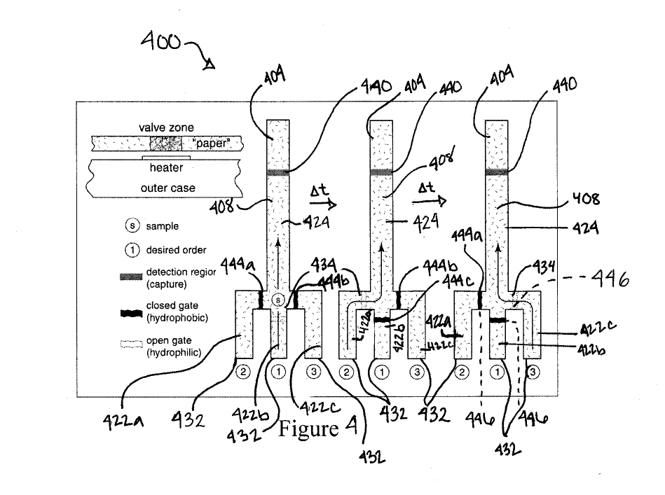

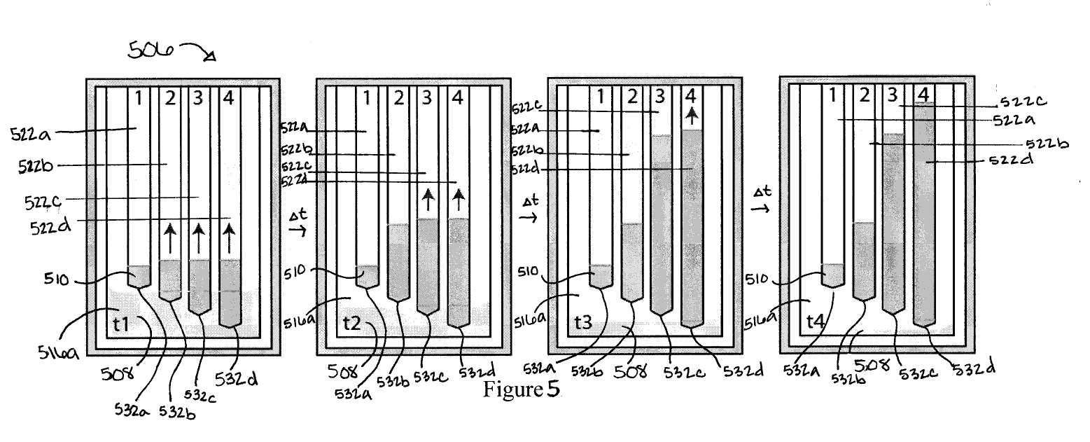

Referring next to FIG. 2D, the pathway 222f has a switchable barrier 244 disposed thereon and positioned to block or hinder fluid flow along the pathway 222f. In one embodiment, the switchable barrier 244 can be composed of a material that is hydrophilic at room temperature and hydrophobic upon heating, thereby altering the time required for fluid to travel the length of the pathway 222f in a heat-dependent manner. In an alternative embodiment, the material is hydrophobic at room temperature and hydrophilic upon heating. The flow-metering element 206d can further include one or more heating elements 246 disposed at one or more locations along or near the pathway 222f. The heating element 246 can be used to effect a switch between hydrophilic and hydrophobic states of a barrier 244 disposed on the wick. Alternatively, the material can switch between hydrophilic and hydrophobic with a change in pH or other property. The time required for fluid to travel the length of the pathway 222f can accordingly be controlled in a heat- or pH-dependent manner. Switchable barriers 244 will be discussed further below with reference to FIG. 4.

As discussed above, barriers can control the rate and volume of fluid delivery downstream of the barrier by serving as a physical blockade to fluid flow. Additionally, barriers can also be used to decrease the local resistance to flow over time. For example, dissolvable barriers 242 could be designed to give a constant flow velocity by decreasing the local resistance to counteract the increase in resistance due to the movement of the fluid front. Switchable barriers 244 can be used to actively change the local resistance. In some embodiments, described in further detail below with reference to FIGS. 3A-4, barriers can be used for precise timing of sequential delivery of sample, wash, and/or reagent to a detection region. While FIGS. 2C and 2D each illustrate only a single barrier in a single pathway, in other embodiments there can be more than one barrier in an individual pathway and/or barriers in multiple pathways within a device.

FIGS. 3A-3D illustrate capillarity-based devices or analyzers utilizing soluble barriers 342 as flow-metering elements 306. FIG. 3A, for example, illustrates a series of time-lapsed front views of a pathway 322a having a flow-metering element 306a configured in accordance with an embodiment of the technology. In this particular embodiment, the flow-metering element 306a includes a soluble material 342a that is deposited on the pathway 322a and dried such that it forms a barrier to fluid wicking during the assay. The solution of dissolvable material 342a can be deposited by various methods, including, for example, manual spotting, dipping the paper strip into the solution, use of striping instruments, and use of contact and non-contact spotting devices such as piezo spotters or pneumatic sprayers. When the assay is started, a fluid 308 wicks from an input end 332 of the pathway 322a up to the barrier 342a, which functions as a dam. The fluid 308 dissolves the soluble material over time and is then free to wick toward an output end 334 of the pathway 322a. The rate of dissolution and the size of the soluble barrier 342a determine the amount of delay time, and this can be different for each pathway 322a.

The soluble barrier 342a may be composed of any dissolvable material that is soluble in the assay fluid, including sugars, salts, gum Arabic, gel material, etc. Also, mixtures of these materials can be used to tune the barrier properties and precisely control fluid flow. For example, mixtures of trehalose (fast dissolving barrier material) and sucrose (slow dissolving barrier material) provide barriers with behavior between the two individual materials. In one embodiment, an absorbent pad (not shown) containing trehalose in water (˜40% by weight) can be used to create a stripe of trehalose across a nitrocellulose wicking strip, which is then allowed to dry overnight. Trehalose is also effective as a protein preservative. The dissolvable materials can be reagents themselves, or reagents stored in dry form within soluble materials, for example a detection probe stored in a sugar matrix. In other embodiments, an inert (i.e., non-reagent) barrier 342 may be desired to prevent premature dissolution of the reagent on the downstream side of the barrier. Dry reagents could also be applied on pads or on the porous wick itself on the upstream side, where they would be able to dissolve into the fluid 308 during the timed dissolution of the soluble barrier 342.

FIG. 3B is a series of time-lapsed front views of a capillarity-based device or analyzer 300 having a plurality of soluble barriers 342 (three are shown in the illustrated embodiment as soluble barriers 342a-c) configured in accordance with an embodiment of the technology. In the illustrated embodiment, the device 300 has a plurality of pathways or legs (three are shown as pathways 322b, 322c, and 322d, and numbered 1-3). Each pathway 322b-d includes an input end 332. An analyte sample S is in the middle leg 322c, labeled “1” and reagent, sample, or wash solutions are in each of legs 2 and 3. To perform particular testing on the sample “S”, it is desirable for the analyzer to first allow the sample to flow into a common channel 324, followed by fluid in leg 2, followed by fluid in leg 3. The assay can be used for ordered fluid commingling in the common channel 324 or for sequential, timed delivery of fluid to a detection region 340. This ordering can be implemented in a single user step by use of soluble barriers.

The device 300 can be placed into a fluid source (or otherwise wetted) which begins fluid wicking from input ends 332 toward the detection region 340. Since leg 1 has no soluble barriers or other flow-metering mechanisms, the sample S is simply wicked toward the detection region 340. Fluid is wicked along both leg 2 and leg 3, but is stopped by the respective soluble barriers 342b and 342c. The soluble barrier 342c of leg 3 is larger than the soluble barrier 342b of leg 2, so the soluble barrier 342c of leg 3 takes a greater time to dissolve. As shown in the second pane, fluid breaks through the soluble barrier 342b in leg 2 while the barrier 342c in leg 3 remains. The fluid from leg 2 is now being wicked along the common channel 324 toward the detection region 340. As shown in the third pane of FIG. 3B, the fluid has sequentially dissolved the barrier 342c in leg 3, allowing the fluid in leg 3 to wick toward the detection region 340. Accordingly, the soluble barriers 342b and 342c can serve to perform the chemical analyses under the pre-set timing constraints.

In some embodiments, the downstream side of a barrier 342 is wetted by other assay fluids and dissolution of the barrier 342 occurs from both sides of the barrier. The two fluids meet within the barrier 342, at which point the two fluids begin to move toward the detection region 340. In the illustrated embodiment, for example, fluid from both upstream and downstream sides of the barriers 342b and 342c in legs 2 and 3 works to dissolve the respective barriers. In other embodiments, a portion of a pathway 322 downstream of the soluble barrier 342 can be pre-wet with buffer to control and/or reduce commingling of fluids. In other embodiments, the device 300 can take on different geometric configurations, legs 322 and barriers 342 can be arranged to deliver fluid in different orders to a common channel 324, and there can be more or fewer legs 322 and/or barriers 342.

FIG. 3C is a series of time-lapsed front views of a flow-metering element 306c having a soluble restrictor 342d configured in accordance with another embodiment of the technology. In the illustrated embodiment, the soluble material 342d is patterned to initially span only a portion of the width of a pathway 322e. Similar to the barriers discussed above with reference to FIGS. 2C, 3A, and 3B, the soluble restrictor 342d serves to limit the rate of fluid flow from an input end 332 toward an output end 334 along a pathway 322e. The difference between the embodiment shown in FIG. 3C and those described above, however, is that restrictors 342d always allow some fluid to pass through the pathway. Thus, flow is only slowed, not entirely stopped, for the time period before the dissolution. As shown in FIG. 3C, for example, the restrictors 342d dissolve over time and an open fluid pathway 322e remains. Fluid is continuously supplied from a fluid source (not shown) to surround the restrictor 342, and dissolution occurs faster than it would in the case of stopped flow.

FIG. 3D is a series of side and front views of flow-metering elements 306d comprising various shapes of soluble barriers and soluble restrictors (collectively “soluble barriers”) 342 deposited on pathways 322 and configured in accordance with further embodiments of the technology. As the illustrated examples indicate, there are countless arrangements of soluble barriers 342, and choosing one simply depends on the timing requirements of the particular assay. In some embodiments, for example, the dissolvable material 342 can be deposited on a separate strip of paper 304 or other support that is then added to create a bridge between two parts of the device. The shape and size of the barrier 342 can be varied to tune the dissolution pattern and fluid 308 breakthrough time. In other embodiments, a plurality of barriers 342 in series or in parallel delays the fluid flow more than if only a single barrier 342 was used. Again, the length and/or width of a barrier 342 also affect the rate of fluid flow.

Referring to the soluble barriers 342 of FIGS. 3A-3D collectively, a significant difference of the use of dissolvable barriers over extending the leg length to create delays in reagent arrival at the detection region is that after dissolution, the reagent can have a high average flow rate that is characteristic of flow in the material at small distances from the fluid source since the overall path length of reagent in the device can be kept small. In contrast, delays created by extension of the leg length will result in a lower average flow rate due to the low flow rates characteristic of flow at large distances from the fluid source.

Larger concentrations of deposited dissolvable material lead to reduced voids and tend to reduce flow to a greater extent than smaller concentrations of the same dissolvable material. For example, saturated sucrose or table sugar creates a nearly impenetrable barrier that stops or greatly slows advance of the fluid, while lower concentrations of sucrose include voids that allow continuous, yet slowed, advance of the fluid through the barrier. Different sugars have different levels of saturation (as a weight percent) and give qualitatively different wetting behavior. For example, barriers created by saturated trehalose or glucose are more easily penetrated than barriers created by saturated solutions of sucrose or table sugar.

The dissolvable materials can also affect the viscosity and surface tension of the assay fluid, and thus influence the flow rate. Different dissolvable materials have different effects on these two properties, and high concentration solutions have the largest effects. Restrictions result in lower concentration compared to barriers that span the width of the leg. Since the surface tension is a critical parameter in the Washburn equation, if the solute changes the surface tension of the fluid, the flow downstream of the barrier or restriction can be different than upstream of the barrier. The effect of surface tension is greatest when the paper downstream of the barrier or restriction is dry, and the effect of surface tension is less when the paper downstream is wetted. The effect of viscosity can be significant in both cases. Additives to the dissolvable material can be used to affect these properties. For example, addition of surfactant can reduce the surface tension.

The delay created by a dissolvable barrier or restriction may be varied in many ways, including the dissolution rate of the dissolvable material, the concentration of the deposited solution of dissolvable material, the total expanse of paper treated with the dissolvable material (i.e., the length of a barrier), and/or the shape of the resulting barrier or restriction. All of the variations described above can be used to create a range of delays in a single device. For example, using only simple sugars (trehalose, glucose, sucrose, and table sugar), delays from seconds to an hour or more can be created. For long delays, evaporation of the fluid can affect the delay timing or even lead to stalling of the fluid when the evaporation rate matches the fluid supply rate. High humidity can be created by enclosing the paper in a device with liquid present.

Dissolvable barriers and restrictions can be used to delay the delivery of a reagent to a common fluid channel, and they can also be used to delay movement of fluid into an upstream or a downstream path. For example, a barrier or restriction can be used to open a pathway to an absorbent pad to increase overall flow, to initiate flow, or to reverse the flow through a leg. In the latter case (reversing the flow), a barrier can be timed to coincide with an upstream absorbent pad reaching its fluid capacity, allowing fluid to reverse direction by flowing into the absorbent pad opened by a dissolvable barrier.

FIG. 4 is a series of time-lapsed front views of a capillarity-based device or analyzer 400 having a plurality of switchable barriers 444a-444c (collectively 444) configured in accordance with an embodiment of the technology. The device 400 has several features generally similar to the device 300 described above with reference to FIG. 3B. Instead of having soluble barriers 342, however, the device 400 includes material 444 capable of switching between hydrophobic and hydrophilic states patterned on pathways 422a-422c (collectively 422) of a wick 404. The material 444 serves as a “gate” to control the timing of fluid flow along the wick 404. In one embodiment, for example, a material (e.g., poly-NiPAAm), can switch between hydrophilic and hydrophobic states via changes in temperature or pH. In other embodiments, other switchable materials can be used. A heating element 446 (shown schematically) positioned near the gates 446 serves as a switch. Independently controlled switches can be used for each liquid and/or leg 422.

In the illustrated embodiment, the device 400 has three gates 444a-444c, one on each of the legs 422. The legs 422 are numbered 1-3 in the order that fluids 408 within the legs 422 should be wicked toward a detection region 440 in order to perform a particular assay. In the first pane, the gates 444a and 444b of legs 2 and 3, respectively are closed, while the gate 444c on leg 1 is open allowing fluid 408 in leg 1 (including a sample S) to wick toward the detection region 440. The heating element 446 is applied to leg 1 and leg 2, which switches the hydrophobic/hydrophilic states of the respective gates 444c and 444a. This action closes leg 1 and opens leg 2, as illustrated in the second pane of FIG. 4. At this time, no additional fluid 408 from leg 1 is being wicked toward the detection region 440, but fluid 408 from leg 2 has started wicking into a common leg 424 toward the detection region 440.

As shown in the third pane of FIG. 4, this process is repeated. In particular, the gate 444a in leg 2 is closed and the gate 444b in leg 3 is opened, stopping fluid 408 from leg 2 and allowing fluid 408 from leg 3. This series of actions allows for the correct volume and timing of fluid 408 to move along the wick 404 in order to perform the particular test. Each fluid 408 can be started and stopped repeatedly throughout a measurement if needed. Control electronics, small batteries, and heating elements are sufficiently simple and inexpensive that this could be done in a disposable device or with an inexpensive external controller. In other embodiments, the device 400 can take on different geometric configurations, legs 422 and barriers 444 can be arranged to deliver fluid in different orders to a common leg 424, and there can be more or fewer legs 422 and/or barriers 444.

### 2. Select Embodiments of Volume-Metering Features

FIGS. 5-8C illustrate various embodiments of volume-metering features configured in accordance with embodiments of the technology. The volume-metering features described below, for example, can be used with the device 100 (FIGS. 1A and 1B) or other suitable capillarity-based devices. The volume-metering features are configured to automatically control or modify a volume of fluid flow along one or more fluid pathways (e.g., pathways 122 of FIGS. 1A and 1B) of a porous wick.

FIGS. 5 and 6 illustrate volume-metering features 506 and 606 that use pathway or leg configuration as an independent control of the shut-off time (and thus volume) of fluid delivered to fluid pathways 522 and 622, respectively. More specifically, the volume-metering features of FIGS. 5 and 6 vary the shut-off time of fluid delivered to a particular pathway by varying the depth that a pathway inlet is submerged into a common fluid well. As fluid from the well wicks into the multiple inlets of the device, the fluid level in the well decreases. When the fluid level falls to a position that is below the bottom of a particular inlet leg, there is no longer a fluidic connection between the well and the input end of the respective pathway, thereby shutting off flow along that particular pathway. This process will occur in sequence for increasing inlet leg lengths.

FIG. 5, for example, is a series of time-lapsed front views of volume-metering feature 506 configured in accordance with an embodiment of the technology. As noted above, the volume-metering feature 506 includes a plurality of pathways 522a-522d (collectively 522) each configured to be submerged to different depths of the fluid well 516. In other embodiments, there may be more or fewer pathways 522 and not all pathways 522 need have different lengths. When the pathways 522 are first placed proximate to the fluid well 516 at time t1, input ends 532b-532d of legs 2-4 are submerged in fluid 508 in the fluid well 516. The input end 532a of leg 1 is not submerged, and in this embodiment is pre-wetted with a sample 510 to be tested. In other embodiments, more or fewer legs 522 can be pre-wetted.

As the legs 522 begin to wick the fluid 508, the fluid level in the well 516 decreases. Shorter legs 522 will lose access to the fluid source 516 earlier than longer legs, as the fluid 508 leaves the well 516 via wicking along the legs 522b-d. At time t2, for example, the fluid level has dropped below the input end 532b of leg 2, and leg 2 no longer wicks fluid 508 from the well 516. At time t3, leg 3 is no longer in contact with the fluid 508 and has accordingly ceased to wick fluid 508 from the well 516, leaving only leg 4 to continue to wick fluid 508 from the well 516. At time t4, enough fluid 508 has been pulled from the well 516 such that the fluid level in the well 516 no longer reaches the input end 532d of leg 4. Accordingly, at time t4 no legs 532 are contacting fluid 508 in the well 516. In this manner, the shut-off time of each leg 522 is pre-set and controlled.

The shut-off timing of multiple inlet legs 522 can be affected by two parameters in addition to the length of the legs 522 submerged in the well 516: (1) the volumetric uptake rate of all legs 522 that are in fluidic contact with the well 516 and (2) the rate that the fluid level drops. These additional parameters can be manipulated to change the shut-off time(s) of a single leg or multiple legs 522. The volumetric uptake rate can be varied by changing the size, flow velocity, or liquid capacity of the wicking material, or a separate wicking channel can be added that is not connected to the other legs 522. In the latter case, this wick can further be used as a means of creating a humidified environment in regions of the device. The rate that the fluid 508 level drops in the well 516 can also be varied independently of varying the volumetric uptake rate of the legs 522. In one example, the rate that the fluid 508 level drops in the well 516 can be varied by changing the cross sectional area of the well 516 along the plane perpendicular to gravity; for a given volumetric uptake rate, wells 516 with large fluid surface areas drop more slowly than wells with small fluid surface area. In another example, additional components, such as a secondary porous wick that absorbs fluid 508 from the well 516, can alter the rate the fluid 508 drops in the well 516. Further, a change in the material or material properties (i.e., surface treatments) can be used to affect both of these parameters and therefore can be used to control the shut-off timing.

FIG. 6 is a series of time-lapsed front views of a capillarity-based device or analyzer 600 having a volume-metering element 606 configured in accordance with another embodiment of the technology. Specifically, the device 600 uses a volume-metering element 606 where a plurality of pathways 622a-622d (collectively 622) on a wick 604 have lengths that are designed to “stage” multiple reagents 614a-614c (collectively 614) in a common leg 624 of the device 600 for sequential delivery to a detection region 640.

Control of the type of reagent 614 that is delivered to the detection region 640 via a particular inlet 622, for example, can be accomplished via spotting of different dried reagents 614 on various legs, either directly on the porous wick 604 or on separate reagent pads (not shown). As fluid 608 from a common well 616 passes onto the input end 632 of a leg 622 having the dried reagent 614, the reagent 614 is reconstituted and flows along the leg 622 and toward an output end 634 into the common leg 624 for sequential delivery to the detection region 640. Reagent delivery can be adjusted such that only one reagent 614 is delivered at a time to the detection region 640 or such that multiple reagents 614 are flowing to the detection region 640 simultaneously in parallel streams, as required by the device application. As in the embodiments discussed above with reference to FIG. 5, inputs 632 of shorter legs 622 stop contacting fluid 608 in the fluid well 616 before inputs 632 of longer legs 622. Accordingly, fluid 608 in shorter legs stops dissolving and delivering reagents 614 before fluid 608 in longer legs 622. Sequential delivery of reagents 614 to the detection region 640 of the device results in the generation of a signal indicating an assay outcome.

In some embodiments, the wick 604 can be composed of a single material in a common fluid well 616. However, in an alternate embodiment, a composite paper network can be composed of multiple materials (with different pore sizes, base material chemistries, and/or surface treatments) for the different inlet legs 622, dry reagent pads 614, main leg 624, detection region 640, etc. These different materials can provide additional flexibility to optimize the dry storage, reconstitution, and delivery of each reagent 614. This can enable more precise control of the integrated sequence of reagent delivery to the detection region 640 of the device. In still further embodiments, the device 600 can include individual wells 616 for each of the inlet legs 622 such that the dimensions and/or fluid level of each well 616 can be varied independently to affect the shut-off timing of the multiple inlet legs 622.

FIG. 7A is a top view of a substrate 754 having volume-metering absorbent pads 718a-718c (collectively 718) configured in accordance with another embodiment of the technology. Referring first to FIG. 7A, individual absorbent pads 718 are placed on storage wicking strips 756a-756c (collectively 756) that arc in fluid contact with reagent storage wells 758. The material and size of the storage strips 756 are chosen such that they can hold and are capable of delivering a volume of fluid to the metering delivery pad 718 that is in excess of the metering pad 718 fluid capacity.

Fluid reagent from the reagent storage wells 758 is wicked via capillary action successively onto the storage wicking strips 756 and then onto the absorbent pads 718. In one embodiment, the absorbent pads 718 become saturated with reagent from the storage strips 756 in a minute or less. In some embodiments, the pads 718 can be on the same substrate 754 as the fluid wicking strips 756, while in other embodiments the pads 718 can be on a separate substrate. In yet another embodiment, the pads 718 can be attached to the storage strip substrate 754 via adhesive, double-stick foam tape, or other attachment mechanism. In still further embodiments, the absorbent pads 718 are supplied with fluid by means other than wicking fluid from a well 758. For example, in one embodiment, fluid is supplied to the absorbent pads 718 by a syringe or pipette, by one or more pads with an excess of fluid, or by dipping the pads into fluid. Multiple pads 718 can be wetted simultaneously. In the illustrated embodiment, three pads 718 are wetted, but there may be more or fewer pads 718 in other embodiments. The pads 718 can be circular, as illustrated, or can be rectangular, triangular, or other shapes. The fluid volume capacity of the individual pads 718 depends on the dimensional characteristics of the pads 718 and the pad material.

FIG. 7B is a top view of the substrate 754 of the capillarity-based device 700 of FIG. 7A. As best seen in FIG. 7B, the substrate 754 can be removeably or fixedly placed on the device 700. In several embodiments, the reagent storage wells 758 and the storage wicking strips 756 are not placed on the device 700 with the substrate 754, as the wells 758 and strips 756 are used for loading the pads 718 with fluid and are not needed during a timed assay. The device 700 can have components generally similar to the device 100 described above with reference to FIGS. 1A and 1B. For example, the device 700 can include pathways or legs 722 configured for wicking fluid from an input end 732 to an output end 734 and converging into a common channel 724 and a detection region 740. The substrate 754 can be aligned with the device 700 such that the saturated absorbent pads 718 are adjacent to, and in fluid communication with, input ends 732 of one or more of the pathways 722. In other embodiments, the individual pads 718, rather than the entire substrate 754, are placed on the device 700. In still further embodiments, the pads 718 and device 700 are proximate to one another on a hinged, creased, or otherwise foldable substrate (not shown) and the pads 718 can be contacted with the device 700 by folding the pads 718 onto the device 700. A fixed volume of reagent flows out of the metering delivery pads into the inlets 732 of the pathways 722. The user (not shown) can control the volume delivered to each inlet 732 of the device 700 by varying the size/material of the individual metering delivery pad 718 associated with that inlet 732. Identical porous pads 718 could be loaded with differing volumes of fluid, or porous pads of differing fluid-carrying capacities could be employed. Porous pads can also be designed and selected on the basis of release properties (e.g., material choice or surface treatment), such that time of fluid entering the input legs 722 is limited by the release capacity of the porous pad 718, independent of the pad's fluid-carrying capacity. This is expected to enable independent control of the total volume and total time of reagent delivery to each inlet 732 and allow for fluid flow within the device 700 to be controlled and automatically shut-off.

In an alternate embodiment, instead of loading the absorbent pads 718 with fluid reagents, the metering delivery pads 718 can be pre-loaded with dried reagents so that, with the exception of the sample input, only water or buffer needs to be added to the device 700 to activate the reagents and begin the chemical processing. In another embodiment, additional pads placed downstream on the legs 722 can have dried reagents which are reconstituted upon contacting water or buffer released by the pads 718. This can remove the added complication of adding different reagents to multiple wells 716. Dried reagents can include buffer salts and/or reacting reagents for sample analyte detection.

FIG. 8A is a series of time-lapsed front views of a capillarity-based device or analyzer 800 configured in accordance with an embodiment of the technology. FIG. 8B is a front view of pre-wetted reagent source pads 818a-818c (collectively 818) for use on the device 800. FIG. 8C is a timeline showing the delivery of reagents from the pre-wetted source pads 818 to a detection region 840. Referring to FIGS. 8A-8C together, the device 800 includes a number of components generally similar to those described above with reference to FIGS. 1A and 1B, including a wick 804 having a plurality of pathways or legs 822a-822c (collectively 822). The pathways 822a-822c each includes an input end 832 and an output end 834. In the illustrated embodiment, the wick 804 comprises a first substrate. A plurality of pre-wetted pads 818 are on a second substrate 854.

The device 800 allows for sequential reagent delivery to the detection region 840 using a network having three staggered inlets 832 to a common channel 824. While in the illustrated embodiment there arc three legs 822 and three pre-wetted pads 818, in other embodiments there can be more or fewer legs 822 and/or pads 818. The device 800 is activated when the second substrate 854 is placed in contact with the wick 804. Specifically, the individual pads 818 are placed in contact with inlets 832 on the individual legs 822. Upon activation, the fluids in the pads are wicked from the input ends 832 toward the detection region 840. Varying volumes of reagent can be introduced into the inlets 832 via the absorbent pads 818. The fluid with the shortest pathway 822c reaches the detection region 840 first and exhausts its fluid source first, while the fluid with the longest pathway 822a takes the longest time to reach the detection region 840 and exhausts its fluid source last. The timing for delivery of multiple fluids (i.e., arrival times and duration of flows) can be varied by changing the path length for fluid travel from each inlet 832 and the volume of fluid applied to each inlet 832. Choice of these parameters, along with the fluid capacity of the materials used will also determine the amount of time the reagent flows can overlap. This can be tailored as needed for the requirements of the specific application.

### 3. Further Embodiments of Capillarity-Based Devices and Methods

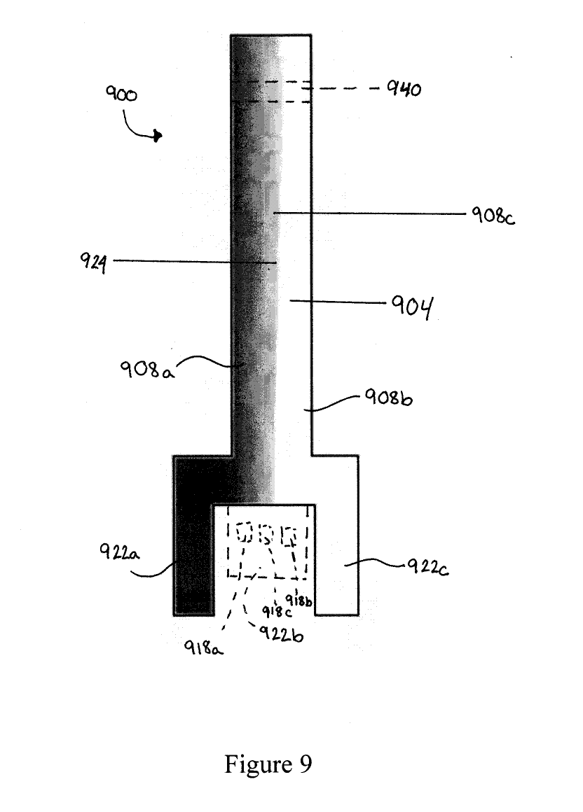

FIG. 9 is a front view of a capillarity-based device or analyzer 900 configured in accordance with an embodiment of the technology. The device has many of the same features disclosed above with reference to FIGS. 1A and 1B, including a wick 904, a plurality of intersecting pathways 922a-922c, and a detection region 940. In the illustrated embodiment, generally parallel side-by-side fluid streams of a first fluid 908a and a second fluid 908b can be wicked within a common channel 924. In the illustrated embodiment, the first fluid 908a and the second fluid 908b are fed into the common channel 924 from separate pathways 922a and 922c, respectively. In some embodiments, separate fluid reservoirs (not shown) supply the fluids 908a and 908b to the device 900. In other embodiments, the first fluid 908a and the second fluid 908b are supplied from fluid source pads (not shown). In still further embodiments, dried and reconstituted source pads (not shown) supply the fluids 908a and 908b.

When two streams 908a and 908b flow by capillary action, they form an interface or diffusion zone 908c across which diffusion occurs. In some embodiments, the diffusion zone 908c can provide information about the contents of the first fluid 908a or the second fluid 908b. In other embodiments, the diffusion zone 908c separates a component from the first fluid 908a or the second fluid 908b. Separation by simple filtration can be used for some components (e.g., cells or particles), and separation by “chromatography” along the length of the device 900 can be used for some components. Other components can be separated from one another based on diffusion between the two streams 908a and 908b. Particles can be separated based on a pH gradient, hydrophobicity, charge, diffusivity of particles, concentration of particles, or other property. Particles can be cells or molecules. Different wick materials can give different quantitative behavior depending on the pore size, membrane chemistry and surface chemistry, and thickness.

In one embodiment, particles or molecules from the first fluid 908a can be separated by diffusion across the interface 908c into the second fluid 908b. Molecules with larger diffusion coefficients are separated from molecules, particles or cells with small diffusion coefficients, for example small analyte molecules into a buffer from a blood sample. The extracted analyte could be recovered by introducing two or more outlet legs to split fluids into two outlet channels (not shown) or by cutting a section of the wick 904. Other separation forces acting across the interface 908c can be implemented for separations using the conditions given above.

In one embodiment, the side-by-side streams 908a and 908b can be used for a diffusion immunoassay (DIA). A DIA is a competitive assay performed using two fluids: (1) a sample spiked with a labeled version of the analyte and (2) a reagent fluid containing a molecule that binds to the analyte. Diffusion between the two parallel streams 908a and 908b permits detection and/or analysis based on diffusivity of particles. Specifically, the analyte diffuses out of the sample stream, where it encounters and binds with the reagent; this binding decreases the diffusion rate of the analyte. High analyte concentrations compete with binding of the labeled version of the analyte, leading to more rapid diffusion of the labeled analyte. The DIA allows quantitative measurement of molecules that are small compared to the binding reagent.

In another embodiment, side-by-side streams can allow multiple samples and/or control samples to be run on a single device 900. In particular, the ability to run control samples in parallel on the same device 900 can greatly improve the effectiveness of controls. Controls can include blanks, positive controls, negative controls, and/or calibration standards. For example, the device 900 can include a sample, a blank (simply a leg without sample or reagent), and a positive control (leg with a known amount of dry analyte that is rehydrated with the buffer). Each of these “samples” flows side-by-side through a detection region 940, followed by application of subsequent steps to the entire detection region 940 (washes, indicators, etc).

In still further embodiments, dry reagents 918a/918b/918c (shown in broken lines) patterned at different locations across the width (perpendicular to flow direction) flow side-by-side and can be delivered to specific detection regions 940. This arrangement is required for assays such as the direct IgM assays: in the direct assay all IgM is captured in the detection region 940, and specific detection is achieved by applying disease-specific detection reagents (typically antigen+antibody) to create detection regions for each analyte. In still other embodiments, a third fluid, such as a buffer or additional reagent, flows between and generally parallel to the first and second fluid streams 908a and 908b. Reagents 918a/918b/918c can enter the common channel 924 from a single central leg 922b (as shown in broken lines) or can enter the common channel 924 from separate legs 922a and 922c. In other embodiments, any number of streams can flow side-by-side in the device 900.

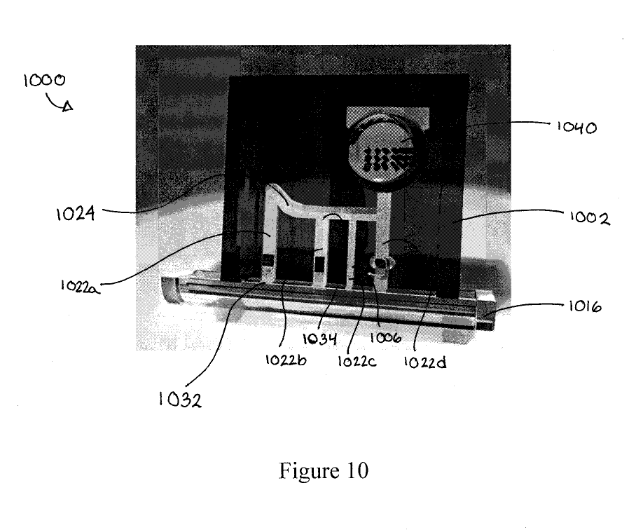

FIG. 10 is an isometric view of a capillarity-based device 1000 configured in accordance with an embodiment of the technology. The device 1000 has several features generally similar to the features of device 100 described with reference to FIGS. 1A and 1B. For example, the device 1000 can include a base 1002 having a fluid well 1016, and a wick 1004 having pathways 1022 capable of wicking fluid from an input end 1032 to an output end 1034, and a detection region 1040. The pathways 1022 are arranged and sized to control fluid flow through the device. Specifically, for a given test, the first leg 1022a takes a serpentine path to join a common channel 1024 of fluid flow in the device. The extended length of this pathway 1022a increases the amount of time required for fluid from the first leg 1022a to reach the detection region 1040 compared to fluid from the other legs 1022b/1022c/1022d. The third leg 1022c has a narrower width than the other legs 1022a/1022b/1022d, serving as a flow-metering device 1006 as discussed above with reference to FIG. 2B.

The capillarity-based devices and analyzers disclosed herein offer several advantages over conventional systems. Slow diffusion is the cause of slow assays in conventional plate formats, and the wick format virtually eliminates this limitation. The wick microfluidic assay arrangement of the devices described above with reference to FIGS. 1A-10 take advantage of high surface area and rapid transport while allowing individual control over flow rates and times for each step of the multi-step assay. Thus, the incubation step can be separately tuned for the sample, each reagent (e.g., secondary antibody, enzyme substrate), and each wash.

Generally, the devices configured in accordance with the present technology adapt the features of microfluidic devices to a porous wick (or paper) system, but without the need for external pumps, mechanical or cicctroosmotic, and without the need for pressure or vacuum sources to regulate the flow of fluid. Thus, no external force is applied to the device to modulate the flow of fluid by means other than the capillary action (surface tension) of the wick and the associated absorbent pads. Additionally, the present technology provides a multi-step chemical process with a single activation step, whereas conventional immunoassays normally involve a series of distinct user steps carried out sequentially. Multi-step assays have several fundamental advantages that lead to increased accuracy and sensitivity: reduced background from washing steps, sensitive detection from enzymatic amplification, and ability to independently optimize each assay step.

The devices disclosed herein are also expected to improve the detection limits for analytes, such as simultaneous detection of two antigens from malarial parasites in blood, but at a manufacturing cost equal to that of conventional rapid diagnostic tests (RDTs). Further, results of a chemical process performed on the device can be read by eye or by cameras of mobile devices. For example, by capturing device detection spot intensities with mobile device cameras, blood antigen concentrations can be rapidly measured locally or remotely. This could greatly aid in screening for the degree of subclinical infections at remote sites. This new approach to point-of-care diagnostics combines the sophistication of chemical processing developed in microfluidics with the simplicity and low cost of lateral flow immunoassays.

From the foregoing it will be appreciated that, although specific embodiments of the technology have been described herein for purposes of illustration, various modifications may be made without deviating from the spirit and scope of the technology. For example, the presence/configuration of the base or housing, the number of pathways, flow-metering elements, volume-metering features, the use of pre-wetted pads, the specific types of fluids, and the material choices for various components of the devices described above with reference to FIGS. 1A-10 may vary in different embodiments of the technology. Further, certain aspects of the new technology described in the context of particular embodiments may be combined or eliminated in other embodiments. For example, in the embodiments illustrated above, various combinations of flow-metering and volume-metering elements or features may be combined into a single device. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

1-43. (canceled)

44. A device for performing chemical processes, the device comprising:
a base;
a porous wick carried by the base, wherein the porous wick comprises a first pathway, a second pathway, and an intersection at which the first pathway and the second pathway converge, wherein each pathway comprises a length defined by an input end and an output end, and wherein the input ends of the first pathway and the second pathway are wettably distinct, and further wherein, upon wetting of the input ends, fluid is configured to travel by capillary action along each pathway;
a first volumetric source in fluid communication with the input end of the first pathway, wherein the first volumetric source is configured to carry a first volume of fluid, and wherein the first volumetric source is configured to supply fluid to the first pathway from an initial time until the first volume of fluid is substantially depleted from the first volumetric source; and
a second volumetric source in fluid communication with the input end of the second pathway, wherein the second volumetric source is wettably distinct from the first volumetric source and is configured to carry a second volume of fluid, and wherein the second volumetric source is configured to supply fluid to the second pathway from the initial time until the second volume of fluid is substantially depleted from the second volumetric source.

45. The device of claim 44, further comprising a soluble barrier along the porous wick, wherein the soluble barrier is an integral component of the porous wick, and wherein the soluble barrier is positioned to modify a rate or volume of fluid flow along the porous wick.

46. The device of claim 44, further comprising a switchable barrier along the porous wick, wherein the switchable barrier is an integral component of the porous wick, and wherein the switchable barrier is positioned to modify a rate or volume of fluid flow along the porous wick.

47. The device of claim 44 wherein a geometric characteristic of at least one of the first or second pathway is configured to modify a rate or volume of fluid flow along the porous wick.

48. The device of claim 44 wherein the porous wick further comprises a third pathway in fluid communication with the intersection, wherein the third pathway has a third length defined by a third input end and a third output end, and wherein the third pathway has a third length less than the length of the second pathway.

49. The device of claim 44 wherein the first volumetric source and the second volumetric source each comprise a pad at least partially saturated with fluid.

50. The device of claim 44 wherein at least one of the first volumetric source and the second volumetric source comprise an individual fluid reservoir.

51. The device of claim 44 wherein the first volumetric source comprises a first material and the second volumetric source comprises a second material different from the first material.

52. The device of claim 44, further comprising a substrate configured to support the first volumetric source and the second volumetric source, wherein the substrate is configured to interface with the porous wick and simultaneously initiate contact between the first volumetric source with the wick and the second volumetric source with the wick.

53. The device of claim 44 wherein a fluid volume capacity of the first volumetric source is different from a fluid volume capacity of the second volumetric source.

54. The device of claim 44 wherein the first and second volumetric sources are proximate to the base on a hinged, creased, or otherwise foldable substrate, and further wherein the first and second volumetric sources are configured to contact the porous wick by folding the first and second volumetric sources onto the wick.

55. The device of claim 44 wherein a fluid release property of the first volumetric source is different from a fluid release property of the second volumetric source.

56. The device of claim 44, further comprising a dried reagent pad in contact with the first pathway.

57. The device of claim 44 wherein the first pathway has a first pathway length and the second pathway has a second pathway length different from the first pathway length.

58. A method for performing a chemical process, the method comprising:
simultaneously wetting an input end of a first pathway with fluid from a first volumetric source containing approximately a first volume of fluid and wetting an input end of a second pathway with fluid from a second volumetric source containing approximately a second volume of fluid, wherein the second volumetric source is wettably distinct from the first volumetric source;
wicking the first volume of fluid along the first pathway from the input end of the first pathway toward an output end of the first pathway;
wicking the second volume of fluid along the second pathway from the input end of the second pathway toward an output end of the second pathway; and
wicking the first volume of fluid and the second volume of fluid along a common pathway to a detection region.

59. The method of claim 58 wherein the first volumetric source has a first volumetric capacity and the second volumetric source has a second volumetric capacity different from the first volumetric capacity.

60. The method of claim 58 wherein the first volumetric source has a first fluid release property and the second volumetric source has a second fluid release property different from the first fluid release property.

61. The method of claim 58 wherein the first volumetric source and the second volumetric source each comprise a fluid reservoir or a porous pad.

62. The method of claim 58, further comprising reconstituting a dried reagent on at least one of the first pathway or the second pathway.
